(19) United States
(12) Patent Application Publication
(10) Pub. No.: US 2024/0355421 A1
(43) Pub. Date: Oct. 24, 2024

(54) METHOD, APPARATUS AND DEVICE FOR IDENTIFYING SOURCE PRIMER OF NONSPECIFIC AMPLICATION SEQUENCE

(71) Applicant: BOE Technology Group Co., Ltd., Beijing (CN)

(72) Inventor: Mengjia Liu, Beijing (CN)

(73) Assignee: BOE Technology Group Co., Ltd., Beijing (CN)

(21) Appl. No.: 18/027,260

(22) PCT Filed: May 27, 2022
(86) PCT No.: PCT/CN2022/095696

§ 371 Date: Mar. 20, 2023

(51) Int. Cl. G16B 30/10

(52) U.S. Cl. CPC G16B 30/10

(57) ABSTRACT

A method, an apparatus and a device for identifying a source primer of a nonspecific amplification sequence are provided in the present disclosure, which belongs to the technical field of gene detection. The method includes: acquiring amplification sequence data of an amplified gene obtained by primer amplification of a target gene fragment, source gene sequence data of a source gene to which the target gene fragment belongs, and primer sequence data used in the primer amplification; aligning the amplification sequence data to the source gene sequence data, and taking the amplification sequence data that does not match the source gene sequence data as nonspecific amplification sequence data; and aligning the nonspecific amplification sequence data to the primer sequence data, and taking a primer with primer sequence data being matched with the nonspecific amplification sequence data as an amplification source primer of the nonspecific amplification sequence.

### TECHNICAL FIELD

The present disclosure belongs to the technical field of gene detection and, more particularly, to a method, an apparatus and device for identifying a source primer of a nonspecific amplification sequence.

### BACKGROUND

In related art, in identifying lymphoma using Next-Generation Sequencing (NGS) technology, it needs to perform multiplexed amplification, high-throughput sequencing and data analysis on DNA (generally on IGH and IGK chains of a B-cell receptor or TCRB, TCRD or other chains of a T-cell receptor) through upstream experiments to identify polyclonal rearrangement of lymphocytes.

However, because V, D and J, which are involved in chain composition, exist in a form of gene clusters on the genome and there are many gene families, polyclonal rearrangement is largely varied and a large number of nonspecific amplification can be easily caused. At present, a proportion of target fragments in multiplexed amplification sequencing data is less than 50%, and low data efficiency caused by nonspecific amplification is not considered in conventional amplification and analysis.

### SUMMARY

A method, an apparatus and a device for identifying a source primer of a nonspecific amplification sequence are provided in the present disclosure.

A method for identifying a source primer of a nonspecific amplification sequence provided in some embodiments of the present disclosure, including:
acquiring amplification sequence data of an amplified gene obtained by primer amplification of a target gene fragment, source gene sequence data of a source gene to which the target gene fragment belongs, and primer sequence data used in the primer amplification;
aligning the amplification sequence data to the source gene sequence data, and taking the amplification sequence data that does not match the source gene sequence data as nonspecific amplification sequence data; and
aligning the nonspecific amplification sequence data to the primer sequence data, and taking a primer with the primer sequence data being matched with the nonspecific amplification sequence data as an amplification source primer of the nonspecific amplification sequence.

Optionally, after aligning the amplification sequence data to the source gene sequence data, and taking the amplification sequence data that does not match the source gene sequence data as the nonspecific amplification sequence data, the method further includes:
aligning the nonspecific amplification sequence data to reference genome sequence data to obtain an alignment result; and
determining position information of source gene of the nonspecific amplification sequence on the genome according to the alignment result.

Optionally, determining the position information of the source gene of the nonspecific amplification sequence on the genome according to the alignment result includes:
counting at least one of a genome source, a sequence position and sequence features of the nonspecific amplification sequence on the reference genome according to distribution and a position of the alignment result on the reference genome sequence data.

Optionally, when the target gene fragment is an immune gene fragment, the gene sequence data includes sequence data with overlapping double-ended sequencing and sequence data with non-overlapping double-ended sequencing;
acquiring the amplification sequence data of the amplified gene obtained by primer amplification of the target gene fragment includes:
acquiring raw data obtained by primer amplification of the gene fragment;
performing an overlapping operation on a first gene fragment and a second gene fragment in the raw data with an overlapping sequence length being greater than or equal to a first sequence length threshold and with a sequence length after overlapping being greater than or equal to a second sequence length threshold, so as to obtain the sequence data with overlapping double-ended sequencing; and
taking amplification sequence data in the raw data with an overlapping sequence length being less than the first sequence length threshold or with a sequence length after overlapping being less than the second sequence length threshold as the sequence data with non-overlapping double-ended sequencing.

Optionally, the source gene sequence data includes sequence data in a V gene family, sequence data in a D gene family and sequence data in a J gene family;
aligning the amplification sequence data to the source gene sequence data, and taking the amplification sequence data that does not match the source gene sequence data as the nonspecific amplification sequence data includes:
aligning the sequence data with overlapping double-ended sequencing and the sequence data with non-overlapping double-ended sequencing to the sequence data in the V gene family, the sequence data in the D gene family and the sequence data in the J gene family, respectively, so as to obtain a consistency alignment value of the sequence data with overlapping double-ended sequencing and the sequence data with non-overlapping double-ended sequencing;
taking a sum of lengths of sequence data in the sequence data with overlapping double-ended sequencing with consistency alignment values to the sequence data in the V gene family, the sequence data in the D gene family and the sequence data in the J gene family being greater than or equal to a consistency alignment value threshold as a comparable length of the sequence data with overlapping double-ended sequencing;
taking the sequence data with overlapping double-ended sequencing with the comparable length being less than a comparable length threshold as the nonspecific amplification sequence data; and
taking sequence data in the sequence data with non-overlapping double-ended sequencing with consistency alignment values to the sequence data in the V gene family, the sequence data in the D gene family and the sequence data in the J gene family being smaller than the consistency alignment value threshold as the nonspecific amplification sequence data.

Optionally, after aligning the amplification sequence data to the source gene sequence data, and taking the amplification sequence data that does not match the source gene sequence data as the nonspecific amplification sequence data, the method further includes:
performing de-redundancy processing on the nonspecific amplification sequence data, so as to remove redundant sequence data of the nonspecific amplification sequence data, the redundant sequence data being sequence data with a proportion of repeated bases in the sequence being greater than or equal to a proportion threshold.

Optionally, before aligning the amplification sequence data to the source gene sequence data, and taking the amplification sequence data that does not match the source gene sequence data as the nonspecific amplification sequence data, the method further includes:
removing low-quality sequence data in the amplification sequence data.

Optionally, removing the low-quality sequence data in the amplification sequence data includes:
removing adapter sequence data with a sequence end length being greater than or equal to an end length threshold in the amplification sequence data after being spliced, and removing sequence data with a sequence average quality value being less than a quality value threshold in the amplification sequence data.

Optionally, removing the low-quality sequence data in the amplification sequence data includes:
removing a low-quality section with the quality value being less than the quality value threshold in the amplification sequence data, and removing the low-quality sequence data with a sequence length being less than a third sequence length threshold in the amplification sequence data after the low-quality section being removed.

An apparatus for identifying a source primer of a nonspecific amplification sequence provided in some embodiments of the present disclosure, including:
an acquisition module configured to acquire amplification sequence data of an amplified gene obtained by primer amplification of a target gene fragment, source gene sequence data of a source gene to which the target gene fragment belongs, and primer sequence data used in the primer amplification; and
an alignment module configured to align the amplification sequence data to the source gene sequence data, and to take the amplification sequence data that does not match the source gene sequence data as nonspecific amplification sequence data; and
align the nonspecific amplification sequence data to the primer sequence data, and take a primer with the primer sequence data being matched with the nonspecific amplification sequence data as an amplification source primer of the nonspecific amplification sequence.

Optionally, the alignment module is further configured for:
aligning the nonspecific amplification sequence data to reference genome sequence data to obtain an alignment result; and
determining position information of source gene of the nonspecific amplification sequence on the genome according to the alignment result.

Optionally, the alignment module is further configured for:
counting at least one of a genome source, a sequence position and sequence features of the nonspecific amplification sequence on the reference genome according to distribution and a position of the alignment result on the reference genome sequence data.

Optionally, when the target gene fragment is an immune gene fragment, the gene sequence data includes sequence data with overlapping double-ended sequencing and sequence data with non-overlapping double-ended sequencing;

Optionally, the acquisition module is further configured for:
acquiring raw data obtained by primer amplification of the gene fragment;
performing an overlapping operation on a first gene fragment and a second gene fragment in the raw data with an overlapping sequence length being greater than or equal to a first sequence length threshold and with a sequence length after overlapping being greater than or equal to a second sequence length threshold, so as to obtain the sequence data with overlapping double-ended sequencing; and
taking amplification sequence data in the raw data with an overlapping sequence length being less than the first sequence length threshold or with a sequence length after overlapping being less than the second sequence length threshold as the sequence data with non-overlapping double-ended sequencing.

Optionally, the source gene sequence data includes sequence data in a V gene family, sequence data in a D gene family and sequence data in a J gene family;

The alignment module is further configured for:
aligning the sequence data with overlapping double-ended sequencing and the sequence data with non-overlapping double-ended sequencing to the sequence data in the V gene family, the sequence data in the D gene family and the sequence data in the J gene family, respectively, so as to obtain a consistency alignment value of the sequence data with overlapping double-ended sequencing and the sequence data with non-overlapping double-ended sequencing;
taking a sum of lengths of sequence data in the sequence data with overlapping double-ended sequencing with consistency alignment values to the sequence data in the V gene family, the sequence data in the D gene family and the sequence data in the J gene family being greater than or equal to a consistency alignment value threshold as a comparable length of the sequence data with overlapping double-ended sequencing;
taking the sequence data with overlapping double-ended sequencing with the comparable length being less than a comparable length threshold as the nonspecific amplification sequence data; and
taking sequence data in the sequence data with non-overlapping double-ended sequencing with consistency alignment values to the sequence data in the V gene family, the sequence data in the D gene family and the sequence data in the J gene family being smaller than the consistency alignment value threshold as the nonspecific amplification sequence data.

Optionally, the alignment module is further configured for:
performing de-redundancy processing on the nonspecific amplification sequence data, so as to remove redundant sequence data of the nonspecific amplification sequence data, the redundant sequence data being sequence data with a proportion of repeated bases in the sequence being greater than or equal to a proportion threshold.

Optionally, the acquisition module is further configured for:
removing low-quality sequence data in the amplification sequence data.

Optionally, the acquisition module is further configured for:
removing adapter sequence data with a sequence end length being greater than or equal to an end length threshold in the amplification sequence data after being spliced, and removing sequence data with a sequence average quality value being less than a quality value threshold in the amplification sequence data.

Optionally, the acquisition module is further configured for:
removing a low-quality section with the quality value being less than the quality value threshold in the amplification sequence data, and removing the low-quality sequence data with a sequence length being less than a third sequence length threshold in the amplification sequence data after the low-quality section being removed.

A computing processing device provided in some embodiments of the present disclosure, including:
a memory with computer-readable code stored therein;
one or more processors, the computing processing device executing the method for identifying the source primer of the nonspecific amplification sequence stated above when the computer-readable code is executed by the one or more processors.

A computer program, including computer-readable code provided in some embodiments of the present disclosure, which, when executed on a computing processing device, causes the computing processing device to execute the method for identifying the source primer of the nonspecific amplification sequence stated above.

A non-transient computer-readable medium provided in some embodiments of the present disclosure with a computer program of the method for identifying the source primer of the nonspecific amplification sequence stated above stored therein.

The above description is only a summary of technical schemes of the present disclosure, which can be implemented according to contents of the specification in order to better understand technical means of the present disclosure; and in order to make above and other objects, features and advantages of the present disclosure more obvious and understandable, detailed description of the present disclosure is particularly provided in the following.

### BRIEF DESCRIPTION OF THE DRAWINGS

In order to explain embodiments of the present disclosure or the technical scheme in the prior art more clearly, the drawings required in the description of the embodiments or the prior art will be briefly introduced below; obviously, the drawings in the following description are some embodiments of the present disclosure, and other drawings can be obtained according to these drawings by those of ordinary skill in the art without paying creative labor.

FIG. 1 schematically shows a flow chart of a method for identifying a source primer of a nonspecific amplification sequence according to some embodiments of the present disclosure;

FIG. 2 schematically shows a flow chart of another method for identifying a source primer of a nonspecific amplification sequence according to some embodiments of the present disclosure;

FIG. 3 schematically shows another flow chart of another method for identifying a source primer of a nonspecific amplification sequence according to some embodiments of the present disclosure;

FIG. 4 schematically shows another flow chart of another method for identifying a source primer of a nonspecific amplification sequence according to some embodiments of the present disclosure;

FIG. 5 schematically shows a schematic diagram showing effect of a method for identifying a source primer of a nonspecific amplification sequence according to some embodiments of the present disclosure;

FIG. 6 schematically shows another schematic diagram showing effect of a method for identifying a source primer of a nonspecific amplification sequence according to some embodiments of the present disclosure;

FIG. 7 schematically shows a structural diagram of an apparatus for identifying a source primer of a nonspecific amplification sequence according to some embodiments of the present disclosure;

FIG. 8 schematically shows a block diagram of a computing processing device for executing the method according to some embodiments of the present disclosure; and

FIG. 9 schematically shows a storage unit for holding or carrying program codes for implementing the method according to some embodiments of the present disclosure.

### DETAILED DESCRIPTION OF THE EMBODIMENTS

In order to make the objects, the technical solutions and the advantages of the embodiments of the present disclosure clearer, the technical solutions of the embodiments of the present disclosure will be clearly and completely described below with reference to the drawings of the embodiments of the present disclosure. Apparently, the described embodiments are merely certain embodiments of the present disclosure, rather than all of the embodiments. All of the other embodiments that a person skilled in the art obtains on the basis of the embodiments of the present disclosure without paying creative work fall within the protection scope of the present disclosure.

Lymphoma is a kind of monoclonal proliferative disease originating from a lymphohematopoietic system. In the past 30 years, incidence rate thereof has increased by 3% to 5% every year, and has doubled around the world. There are 100,000 new cases of lymphoma in China every year, with an annual growth rate of more than 5%, which is one of the most rapidly growing common malignant tumors. According to a global cancer statistics report in 2020, there are about 600,000 new cases of lymphoma every year, accounting for 55% of all hematological tumors. Accurate diagnosis and classification of lymphoma is a key to its treatment and prognosis. Molecular genetic characteristics can supplement information that ordinary pathological examination cannot provide, and become an important means to distinguish subtypes.

NGS technology is the next generation sequencing technology, also known as high-throughput sequencing, which is characterized by high throughput and high resolution and can read hundreds of thousands to millions of DNA molecules in parallel at one time. It can provide rich genetic information, while greatly reducing sequencing cost and shortening sequencing time. With application of the NGS technology, molecular diagnosis has gradually begun to function in accurate diagnosis of lymphoma and other diseases, which can help clinicians to better diagnose lymphoma, choose treatment schemes, determine prognosis, and detect residual micro-lesions.

The NGS technology has following advantages when applied to detection and identification of lymphoma: 1) high sensitivity, which can reach 10-6, which is 100 times higher than that of traditional flow cytometry. 2) Individualized detection, due to great differences among individuals in an immune group, individual VDJ rearrangement can be identified by sequencing analysis; 3) tracking of new clones, with development of diseases and medication, individuals can be subjected to clone evolution, and new clones can be tracked so as to give patients more accurate test results; 4) determination of the prognosis, it can be generally determined whether the prognosis is good by hypermutation of IGHV. When a mutation ratio of IGHV is greater than 2%, the prognosis is considered to be good, and such accurate mutation results can guide clinicians to adopt individualized treatment solutions for patients.

In related art, in identifying lymphoma using the NGS technology, it needs to perform multiplexed amplification, high-throughput sequencing and data analysis on DNA (generally on IGH and IGK chains of a B-cell receptor or TCRB, TCRD or other chains of a T-cell receptor) through upstream experiments to identify clonal rearrangement of lymphocytes. However, because V, D and J, which are involved in chain composition, exist in a form of gene clusters on a source gene, and there are many gene families, rearrangement is largely varied, in which a large number of nonspecific amplification can be caused. At present, a proportion of target fragments in the sequence data is less than 50%. Low data efficiency caused by nonspecific amplification is not considered in conventional amplification and analysis. In order to optimize effect of primer amplification and improve data efficiency, it is necessary to analyze a nonspecific amplification result so as to determine a problem caused by the primer amplification and provide a direction for subsequent primer optimization.

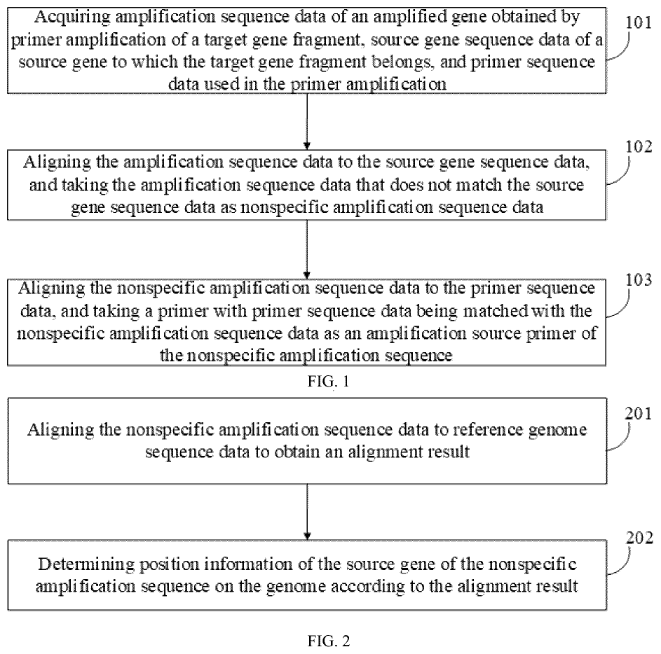

FIG. 1 schematically shows a flow chart of a method for identifying a source primer of a nonspecific amplification sequence according to the present disclosure, which includes following steps 101 to 103.

In the step 101, amplification sequence data of an amplified gene obtained by primer amplification of a target gene fragment, source gene sequence data of a source gene to which the target gene fragment belongs, and primer sequence data used in the primer amplification are acquired.

It should be noted that the target gene fragment is a characteristic gene fragment in DNA (DeoxyriboNucleic Acid) or RNA (Ribonucleic Acid) of an organism which is multiplexed amplified using primers in upstream experiments. The amplification sequence data is data obtained by high-throughput sequencing of the target gene fragment. The source gene sequence data is data obtained by high-throughput sequencing of the source gene from which the target gene fragment originates, which can be directly extracted in actual use by communicating with a source gene database by sequencing a source gene from which recombination of primer amplified target chains originates. The primer is a short segment of single-stranded DNA or RNA, which is a polynucleotide chain acting as a starting point for extension of a respective polynucleotide chain in a nucleic acid synthesis reaction. Because the primer is designed in advance, primer genes can be amplified to construct a primer database, and the primer sequence data can be directly extracted from the primer database when used.

Optionally, an execution subject of some embodiments of the present disclosure may be a service end or a terminal that performs analysis on amplification sequence data, and the service end or terminal may be an electronic device with data processing and data transmission functions such as a server, a personal computer, a tablet, a notebook, etc. In the following, schemes of the present disclosure will be described in detail with the service end as the execution subject as an example, and of course, the execution subject of some embodiments of the present disclosure may also be other types of electronic devices, which can be set according to actual needs and is not limited herein.

In the embodiment of the present disclosure, in the upstream experiments, the target gene fragment can be subjected to multiplexed amplification using a PCR technology based on a primer designed for the target gene fragment so as to obtain an amplified gene, and the amplified gene and the source gene from which the target gene fragment originates can be subjected to high-throughput sequencing, so as to obtain the amplification sequence data of the amplified gene and the source gene sequence data of the source gene.

In practical applications, the operator can input the amplification sequence data into the service end, and the service end will automatically extract the source gene sequence data from the source gene database and the primer sequence data from the primer database, so as to trigger to execute steps of method for identifying the source primer of the nonspecific amplification sequence according to the present disclosure to identify the source primer of the nonspecific amplification sequence in the amplification gene other than expected specific amplification gene.

It can be understood that the multiplexed amplification is usually carried out through primer induction for the target gene fragment. A gene induced and amplified by the primer can be the specific amplification gene, while a gene induced and amplified by the primer beyond expectation can be nonspecific amplification sequence. The nonspecific amplification sequence will not only interfere with subsequent identification and analysis of sequence data, but also consume a lot of experimental resources, greatly reducing effect of gene-specific amplification.

In the step 102, the amplification sequence data is aligned to the source gene sequence data, and the amplification sequence data that does not match the source gene sequence data is taken as nonspecific amplification sequence data.

In the embodiment of the present disclosure, because the target gene fragment from which the amplification sequence data originate is the source gene, the specific amplification gene obtained by amplifying the target gene fragment can be aligned to the source gene sequence data. After the alignment, data in the amplification sequence data with a large aligned length to the source gene sequence data can be regarded as the specific amplification sequence data and data in the amplification sequence data with a small aligned length to the source gene sequence data can be regarded as the nonspecific amplification sequence data, for further analyzing the source primer of the nonspecific amplification sequence data.

In the step 103, the nonspecific amplification sequence data is aligned to the primer sequence data, and a primer with primer sequence data being matched with the nonspecific amplification sequence data is taken as the amplification source primer of the nonspecific amplification sequence.

In the embodiment of the present disclosure, although the nonspecific amplification sequence cannot be aligned to the source gene of the target gene fragment, the nonspecific amplification sequence is obtained by primer-induced recombination amplification, so the nonspecific amplification sequence can be aligned to the primer sequence data of the used primer. It can be understood that the amplification sequence data obtained by primer-induced recombination amplification can all be theoretically aligned to the primer sequence data, and the present disclosure uses this characteristic to identify the primer sequence data that induces and recombines to generate the nonspecific amplification sequence by aligning the nonspecific amplification sequence data in the amplification sequence data to the primer sequence data, so that a primer corresponding to the used primer sequence data can be taken as the amplification source primer of the nonspecific amplification sequence.

In practical applications, after analyzing the amplification source primer corresponding to the nonspecific amplification sequence data, the service end can output the primer sequence data of the amplification source primer and a position and distribution of the nonspecific amplification sequence data for operators to check effect of analyzing the primer, so as to optimize setting of the primer with reference to an output result and improve amplification effect of the primer.

In the embodiment of the present disclosure, the amplification sequence data of the amplified gene is aligned to the source gene sequence data from which the primer amplification target gene fragment originates to screen out the nonspecific amplification sequence data in the amplification sequence data, and then the nonspecific amplification sequence data is aligned to the primer sequence data of the primer used in amplification to screen out the primer sequence data matched with the nonspecific amplification sequence data, so that the amplification source primer of the nonspecific amplification sequence in the amplification sequence can be accurately identified.

Optionally, referring to FIG. 2, after the step 102, the method further includes following steps 201 and 202.

In the step 201, the nonspecific amplification sequence data is aligned to reference genome sequence data to obtain an alignment result.

It should be noted that the reference genome sequence data can be obtained by establishing a database with GRCh38 version of human genome and formatting it with makeblastdb (which is a format conversion tool), and the formatted reference gene database is named GRCh38-db. Of course, the reference genome sequence data can also use other available human genome sequence data, which is only an example here, and can be set according to actual needs and is not limited herein.

In the embodiment of the present disclosure, considering that the nonspecific amplification sequence is obtained by recombination and amplification, with low readability of its base distribution, it is impossible to directly identify a gene sequence of the nonspecific amplification sequence on the source gene via the nonspecific amplification sequence data. Therefore, in the present disclosure, the nonspecific amplification sequence data is aligned to the reference genome sequence data, and a gene sequence fragment of the reference genome sequence data that matches the nonspecific amplification sequence data is determined according to the alignment result.

In the step 202, position information of the source gene of the nonspecific amplification sequence on the genome is determined according to the alignment result.

In the embodiment of the present disclosure, the service end can determine position information of the source gene sequence of the specific amplification gene on the genome according to a position of reference gene sequence data matched with the specific amplification sequence data in the reference genome sequence data, determined according to the alignment result.

Optionally, the step 202 includes counting at least one of a genome source, a sequence position and sequence features of the nonspecific amplification sequence on the reference genome according to distribution and a position of the alignment result on the reference genome sequence data.

In the embodiment of the present disclosure, the nonspecific amplification sequence data is aligned to the reference genome sequence data, and a criterion of sequence alignment is that identity (which indicates a consistency value) is larger than a sequence consistency threshold, that is, a sequence aligned length is greater than or equal to any value from 80% to 95%, such as 80%, 85% or 95%, of a total sequence length. A case where the sequence source position is identified is counted and analyzed, and what is counted includes a genome source with optimal alignment for respective sequences, a specific genome position where the sequence source is mainly concentrated or sequence features.

In the embodiment of the present disclosure, the nonspecific amplification sequence data is aligned to the reference genome sequence data, and the position information of the source gene of the nonspecific amplification sequence on the genome is determined according to the alignment result, so that a problem that a position of a base sequence of the nonspecific amplification sequence is not easy to be identified is solved, and accuracy of identifying the position information of the source gene of the nonspecific amplification sequence is improved.

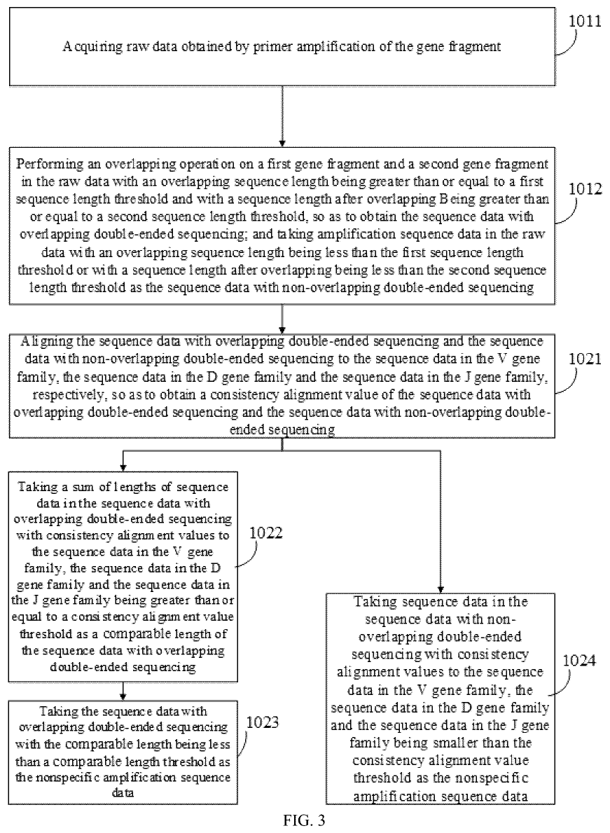

Optionally, when the target gene fragment is an immune gene fragment, the gene sequence data includes sequence data with overlapping double-ended sequencing and sequence data with non-overlapping double-ended sequencing. Referring to FIG. 3, the step 101 includes following steps 1011 and 1012.

In the step 1011, raw data obtained by primer amplification of the gene fragment is acquired.

In the embodiment of the present disclosure, in order to improve identifying accuracy of the nonspecific amplification sequence data, after the service end acquires the raw data of the amplified gene, it can perform an overlapping operation on the raw data, the overlapping operation refers to combining two sequence data in the raw data in a base pairing manner.

In the step 1012, the overlapping operation is performed on a first gene fragment and a second gene fragment in the raw data with an overlapping sequence length being greater than or equal to a first sequence length threshold and with a sequence length after overlapping being greater than or equal to a second sequence length threshold, so as to obtain the sequence data with overlapping double-ended sequencing; and amplification sequence data in the raw data with an overlapping sequence length being less than the first sequence length threshold or with a sequence length after overlapping being less than the second sequence length threshold is taken as the sequence data with non-overlapping double-ended sequencing.

In an embodiment of the present disclosure, the first gene fragment R1 and the second gene fragment R2 may be two different gene fragments which are paired in original amplification gene sequencing data, respectively. A criterion for the overlapping operation is that an overlapping length of R1 and R2 is greater than or equal to a first sequence length threshold, and the first sequence length threshold can be any value from 10 bp to 20 bp, such as 10 bp, 15 bp and 20 bp, and an overlapped sequence length thereof is greater than or equal to the second sequence length threshold, and the second sequence length threshold can be any value from 100 bp to 150 bp, such as 100 bp, 125 bp and 150 bp. Therefore, it is possible to overlap the raw data that meets the overlapping criteria to obtain the sequence data with overlapping double-ended sequencing, and the raw data that do not meet the overlapping criteria can be used as the sequence data with non-overlapping double-ended sequencing. The service end can take the sequence data with overlapping double-ended sequencing and the sequence data with non-overlapping double-ended sequencing as the amplification sequence data for subsequent analysis.

In the embodiment of the present disclosure, the amplification sequence data are overlapped, so that two-way alignment of the sequence data with overlapping double-ended sequencing can be realized for the amplification sequence data in a subsequent alignment process, with a higher accuracy of the alignment result than that of the non-overlapped gene sequence data.

Optionally, the source gene sequence data includes sequence data in a V gene family, sequence data in a D gene family and sequence data in a J gene family. Referring to FIG. 3, the step 102 includes following steps 1021 to 1024.

In the step 1021, the sequence data with overlapping double-ended sequencing and the sequence data with non-overlapping double-ended sequencing are aligned to the sequence data in the V gene family, the sequence data in the D gene family and the sequence data in the J gene family, respectively, so as to obtain a consistency alignment value of the sequence data with overlapping double-ended sequencing and the sequence data with non-overlapping double-ended sequencing.

In the step 1022, a sum of lengths of sequence data in the sequence data with overlapping double-ended sequencing with consistency alignment values to the sequence data in the V gene family, the sequence data in the D gene family and the sequence data in the J gene family being greater than or equal to the consistency alignment value threshold is taken as a comparable length of the sequence data with overlapping double-ended sequencing.

In the step 1023, the sequence data with overlapping double-ended sequencing with a comparable length less than a comparable length threshold is taken as the nonspecific amplification sequence data.

In the step 1024, sequence data in the sequence data with non-overlapping double-ended sequencing with consistency alignment values to the sequence data in the V gene family, the sequence data in the D gene family and the sequence data in the J gene family being smaller than the consistency alignment value threshold is taken as the nonspecific amplification sequence data.

It should be noted that the V, D and J gene families are three main gene families of a reproductive gene sequence of a human germ cell, and these three gene families are distributed in the reproductive gene sequence successively in an order of V, D and J.

In the embodiment of the present disclosure, for an assembled set (sequence data with overlapping double-ended sequencing), “mapped” (completely matched data) can be defined as alignment identity being greater than or equal to the consistency alignment value threshold which can be any value from 80 to 90, such as 80, 85 and 90, and an aligned length of the sequence data with overlapping double-ended sequencing to the V, D and J gene families is greater than or equal to any value from 80% to 90% of a total sequence length, such as 80%, 85% and 90%; “partial mapped” (partially matched data) can be defined as the alignment identity being greater than or equal to the consistency alignment value, and an aligned length of the sequence data with overlapping double-ended sequencing to the V, D and J gene families is greater than or equal to any value from 10% to 20% of the total sequence length, such as 10%, 15% and 20%; and “unmapped” (unmatched data) can be defined as the alignment identity being greater than or equal to the consistency alignment value, and an aligned length of the sequence data with overlapping double-ended sequencing to the V, D and J gene families is smaller than the comparable length threshold which can be any value from 10% to 20% of the total sequence length, such as 10%, 15% and 20%.

For an unassembled set (sequence data with non-overlapping double-ended sequencing), “mapped” is defined as the alignment identity being greater than or equal to the consistency alignment value threshold, and R1 and R2 can be aligned to the V gene family and the J gene family respectively, or one of R1 and R2 can be aligned to the V and J gene families at a same time; “partial mapped” is defined as the alignment identity being greater than or equal to the consistency alignment value threshold, and R1 or R2 can be aligned to one of the gene families V, D and J of immune genes; and “unmapped” is defined as the alignment identity being greater than or equal to the consistency alignment value threshold, R1 or R2 can't be aligned to any one of the gene families V, D and J of immune genes, that is, a consistency alignment value of the sequence data with non-overlapping double-ended sequencing to any one of the gene families V, D and J is less than the consistency alignment value threshold.

Data extracted from mapped, partial mapped and unmapped data sets are stored in a Fasta format, and data amount of the data sets is counted.

It is worth noted that a reason why only the sequence data with non-overlapping double-ended sequencing is aligned to the V and J gene families of immune genes is that the sequence data with non-overlapping double-ended sequencing cannot be aligned to the D gene family in a middle part of an immune gene sequence due to a middle part thereof being not overlapped.

In the embodiment of the present disclosure, only unmapped data after the above alignment to the immune gene, that is, the gene sequence data which is not aligned, is taken as the nonspecific amplification sequence data.

In the embodiment of the present disclosure, referring to above description, the gene sequence data, with a sequence length being less than the comparable length threshold and with an alignment consistency value to immune gene sequence data being greater than the alignment value threshold, of the sequence data with overlapping double-ended sequencing and the sequence data with non-overlapping double-ended sequencing can be taken as the nonspecific amplification sequence data for further analyzing an amplification primer source of the nonspecific amplification sequence.

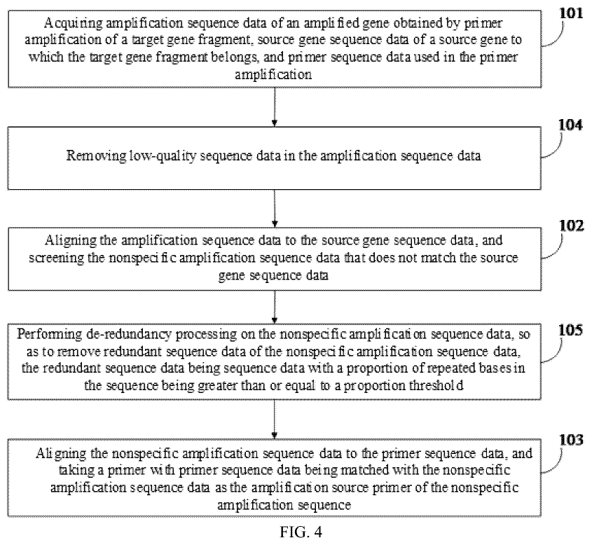

Optionally, referring to FIG. 4, before the step 102, the method further includes step 104.

In the step 104, low-quality sequence data in the amplification sequence data is removed.

According to the embodiment of the present disclosure, the low-quality sequence data in the amplification sequence is removed, so that interference of the low-quality sequence data in a subsequent analysis process to the analysis process and processing resources required for analysis are reduced, and efficiency and accuracy of data analysis are improved.

Optionally, referring to FIG. 4, after the step 102, the method further includes following step 105.

In the step 105, de-redundancy processing is performed on the nonspecific amplification sequence data, so as to remove redundant sequence data of the nonspecific amplification sequence data. The redundant sequence data is sequence data with a proportion of repeated bases in the sequence being greater than or equal to a proportion threshold.

In the embodiment of the present disclosure, the nonspecific amplification sequence data can be subjected to de-redundancy process. Redundant sequences are defined as sequences with sequence similarity identified by using global sequences greater than or equal to a similarity threshold, which can be calculated by dividing a number of identical bases in a sequence by a full length of a shorter sequence. Cd-hit or other similarity identification tools can be used for de-redundancy processing and sequence clustering. Cluster information of nonspecific amplification sequence data after each de-redundancy operation is stored, that is, each clustered sequence in each nonspecific amplification sequence data, its sequence information and its number of sequence are stored, thus reducing a memory occupied by the nonspecific amplification sequence data and time consumption required for subsequent analysis.

Optionally, the step 202 includes removing adapter sequence data with a sequence end length being greater than or equal to an end length threshold in the amplification sequence data after being spliced, and removing sequence data with a sequence average quality value being less than a quality value threshold in the amplification sequence data.

In the embodiment of the present disclosure, the adapter sequence is a short synthetic DNA fragment that contains an enzyme cutting site and can be matched with a blunt end or a sticky end.

In the embodiment of the present disclosure, the amplification sequence data are subjected to be spliced and low-quality data filter, an adapter sequence is identified in the sequencing data according to a sequencing-platform adapter sequence, and the adapter sequence is removed. The removing criteria is that the adapter sequence data is with a sequence end being longer than or equal to the end length threshold which can be any value from 3 bp to 6 bp, such as 3 bp, 4 bp and 6 bp. Then, the low-quality sequence is filtered and removed, and a sequence filtering criteria is that an average quality value of the sequence is less than a quality average threshold, and data amount before and after removing the adapter sequence is counted, and the quality average threshold can be any value from 20 to 25, such as 21, 23 and 25.

Optionally, the step 202 includes removing a low-quality section with the quality value being less than the quality value threshold in the amplification sequence data, and removing the low-quality sequence data with a sequence length being less than a third sequence length threshold in the amplification sequence data after the low-quality section being removed.

In the embodiment of the present disclosure, the low-quality segment in the amplification sequence is removed, with a removing criterion that its quality value is less than a quality value threshold, the quality value threshold can be any value from 20 to 25, for example, 20, 23 and 25, and then the sequence length is filtered, with a filtration criterion that a length of sequence data after the low-quality sequence being removed is less than the third sequence length threshold, and the third sequence length threshold can be any value from 40 bp to 50 bp, such as 40 bp, 45 bp and 50 bp. Quality value and data amount after length filtration are counted.

In the embodiment of the present disclosure, the low-quality sequence data is filtered before analyzing the amplification sequence data, thereby thus reducing the memory occupied by the nonspecific amplification sequence data and time consumption required for subsequent analysis.

Illustratively, in the present disclosure, an embodiment is provided in which the sequence data of a TCRD chain of the multiplexed amplification sequence is taken as the amplification sequence data.

In S1, sequence data is preprocessed, including removing adapters and low-quality sequences, and overlapping and classifying Read.

TCRD chains of 22 samples were amplified and sequenced, with a sequencing strategy of PE150 (pair sequencing, with a Read length of 150 bp). Sequencing data amount of the samples varies from 0.1 M to 0.3 M Reads, as shown in Table 1.

TABLE 1

| Sample | Read Number | Base Count |
|---|---|---|
| 1_1 | 172504 | 25632527 |
| 1_2 | 172504 | 25632781 |
| 2_1 | 189604 | 28265123 |
| 2_2 | 189604 | 28264855 |
| 3_1 | 190496 | 28384842 |
| 3_2 | 190496 | 28386622 |
| 4_1 | 188559 | 28155592 |
| 4_2 | 188559 | 28158643 |
| 5_1 | 1064069 | 156902721 |
| 5_2 | 1064069 | 156934092 |
| 6_1 | 213810 | 31797231 |
| 6_2 | 213810 | 31799872 |
| 7_1 | 153036 | 22709021 |
| 7_2 | 153036 | 22711590 |
| 8_1 | 121329 | 18072029 |
| 8_2 | 121329 | 18074166 |
| 9_1 | 161618 | 24044457 |
| 9_2 | 161618 | 24042931 |
| 10_1 | 193020 | 28411409 |
| 10_2 | 193020 | 28414033 |
| 11_1 | 139463 | 20837756 |
| 11_2 | 139463 | 20834894 |
| 12_1 | 252720 | 37856574 |
| 12_2 | 252720 | 37853502 |
| 13_1 | 119669 | 17814540 |
| 13_2 | 119669 | 17813365 |
| 14_1 | 148860 | 22301210 |
| 14_2 | 148860 | 22298937 |
| 15_1 | 104044 | 15530179 |
| 15_2 | 104044 | 15529233 |
| 16_1 | 140993 | 21037806 |
| 16_2 | 140993 | 21035118 |
| 17_1 | 164674 | 24441146 |
| 17_2 | 164674 | 24443341 |
| 18_1 | 150338 | 22531472 |
| 18_2 | 150338 | 22526562 |
| 19_1 | 81633 | 12110490 |
| 19_2 | 81633 | 12111653 |
| 20_1 | 144494 | 21486430 |
| 20_2 | 144494 | 21486074 |
| 21_1 | 172669 | 25767958 |
| 21_2 | 172669 | 25771119 |
| 22_1 | 146394 | 21821713 |
| 22_2 | 146394 | 21823055 |

wherein “_1” and “2” respectively represent R1 and R2 of paired Reads; “Read Number” indicates a number of sample Reads; “Base Count” indicates a number of bases in a sample.

The adapter sequence is filtered, and a removing criterion is that the adapter is with a sequence end being longer than or equal to 3 bp. The low-quality sequence is filtered and removed, and a sequence filtering criterion is that an average quality value of the sequence is less than 25; the low-quality section in the sequence is removed, and the removing criterion is that its quality value is less than 25; and filtering is performed by the sequence length, and the filtering criterion is that its length after the low-quality sequence is removed is less than 50 bp. Content, a quality value and data amount after length filtration of the adapter sequence are counted, which can be referred to Table 2.

TABLE 2

| | adapter | Qual Filter | | Length Filter | | |
|---|---|---|---|---|---|---|
| Sample | Count | base(Ratio) | | Count(Ratio) | Left Base | Overlapped |
| 1_1 | 0.50% | 247,300 | bp (1.0%) | 3151 (1.83%) | 25013574 | 91.62% |
| 1_2 | 0.40% | 534,757 | bp (2.1%) | | 25003650 | |
| 2_1 | 0.40% | 247,239 | bp (0.9%) | 3087 (1.63%) | 27662761 | 91.87% |
| 2_2 | 0.40% | 524,705 | bp (1.9%) | | 27653561 | |
| 3_1 | 0.40% | 237,367 | bp (0.8%) | 3099 (1.63%) | 27786335 | 92.49% |
| 3_2 | 0.40% | 521,910 | bp (1.8%) | | 27780186 | |
| 4_1 | 0.40% | 236,310 | bp (0.8%) | 2601 (1.38%) | 27616961 | 93.23% |
| 4_2 | 0.40% | 495,149 | bp (1.8%) | | 27585655 | |
| 5_1 | 0.70% | 1,716,550 | bp (1.1%) | 20068 (1.89%) | 153362891 | 92.16% |
| 5_2 | 0.60% | 2,966,251 | bp (1.9%) | | 153211369 | |
| 6_1 | 0.50% | 281,679 | bp (0.9%) | 3316 (1.55%) | 31137888 | 91.91% |
| 6_2 | 0.50% | 587,220 | bp (1.8%) | | 31107676 | |
| 7_1 | 0.90% | 183,958 | bp (0.8%) | 2227 (1.46%) | 22268238 | 92.16% |
| 7_2 | 0.80% | 384,810 | bp (1.7%) | | 22254394 | |
| 8_1 | 0.60% | 164,972 | bp (0.9%) | 2166 (1.79%) | 17660787 | 91.06% |
| 8_2 | 0.50% | 372,466 | bp (2.1%) | | 17638376 | |
| 9_1 | 0.60% | 216,591 | bp (0.9%) | 2663 (1.65%) | 23521084 | 88.44% |
| 9_2 | 0.50% | 457,651 | bp (1.9%) | | 23508381 | |
| 10_1 | 0.60% | 274,743 | bp (1.0%) | 2821 (1.46%) | 27822732 | 92.50% |
| 10_2 | 0.50% | 488,791 | bp (1.7%) | | 27820696 | |
| 11_1 | 2.60% | 168,828 | bp (0.8%) | 2370 (1.70%) | 20386160 | 91.05% |
| 11_2 | 3.20% | 403,955 | bp (1.9%) | | 20362464 | |
| 12_1 | 0.30% | 316,815 | bp (0.8%) | 4141 (1.64%) | 37061577 | 93.06% |
| 12_2 | 0.30% | 708,138 | bp (1.9%) | | 37032102 | |
| 13_1 | 0.40% | 132,282 | bp (0.7%) | 1601 (1.34%) | 17496635 | 90.82% |
| 13_2 | 0.30% | 289,977 | bp (1.6%) | | 17480251 | |
| 14_1 | 0.30% | 168,864 | bp (0.8%) | 2203 (1.48%) | 21880197 | 92.24% |
| 14_2 | 0.30% | 367,573 | bp (1.6%) | | 21874186 | |
| 15_1 | 1.00% | 116,313 | bp (0.7%) | 1763 (1.69%) | 15204782 | 92.41% |
| 15_2 | 1.20% | 298,457 | bp (1.9%) | | 15190462 | |
| 16_1 | 0.50% | 187,406 | bp (0.9%) | 2783 (1.97%) | 20515163 | 88.41% |
| 16_2 | 0.40% | 462,021 | bp (2.2%) | | 20503648 | |
| 17_1 | 0.70% | 235,632 | bp (1.0%) | 3109 (1.89%) | 23843381 | 90.07% |
| 17_2 | 0.50% | 555,955 | bp (2.3%) | | 23807223 | |
| 18_1 | 0.40% | 185,128 | bp (0.8%) | 2656 (1.77%) | 22036503 | 90.56% |
| 18_2 | 0.20% | 445,391 | bp (2.0%) | | 22022538 | |
| 19_1 | 0.90% | 117,633 | bp (1.0%) | 1279 (1.57%) | 11847328 | 87.26% |
| 19_2 | 0.90% | 230,334 | bp (1.9%) | | 11841064 | |
| 20_1 | 0.40% | 232,385 | bp (1.1%) | 2911 (2.01%) | 20917946 | 91.30% |
| 20_2 | 0.30% | 465,195 | bp (2.2%) | | 20918750 | |
| 21_1 | 0.70% | 241,530 | bp (0.9%) | 3621 (2.10%) | 25102673 | 92.00% |
| 21_2 | 0.90% | 602,945 | bp (2.3%) | | 25068179 | |
| 22_1 | 0.50% | 176,417 | bp (0.8%) | 2002 (1.37%) | 21409785 | 90.63% |

Wherein “adapter Count” indicates adapter sequence content in the sequence: “Qual Filter base (Ratio)” indicates a number of bases after quality value filtration (a ratio of filtered bases); “Length Filter Count (Ratio)” indicates a number of filtered sequences of R1 or R2 which does not meet length requirements (a ration of sequences filtered); “Left Base” indicates data volume of remaining bases; and “Overlapped” indicates a ration of sequences that can be overlapped.

Overlapping operation is performed on the R1 end and the R2 end of the sequencing data. An overlapping criterion is that an overlap length of R1 and R2 is greater than or equal to 10 bp, and a sequence length after the overlapping operation is greater than or equal to 100 bp. Overlapping sequences are saved to the assembled set, and a non-overlapping part is saved to the “unassembled” set. Data volume of the two data sets is counted, which can be referred to Table 3.

TABLE 3

| | | | Assembled | | | | Unassembled | |
|---|---|---|---|---|---|---|---|---|
| | Assembled | | Unmapped | | Unassembled | | Unmapped | |
| | Read | Read | Read | Read | Read | Read | Read | Read |
| Sample | Number | Base | Number | Base | Number | Base | Number | Base |
| 1 | 155,157 | 28,452,146 | 53,926 | 10,128,337 | 28,392 | 4,071,425 | 27,746 | 3,990,311 |
| 2 | 171,345 | 32,245,012 | 41,424 | 7,832,836 | 30,344 | 4,378,358 | 29,422 | 4,259,998 |
| 3 | 173,317 | 32,324,586 | 43,196 | 8,098,268 | 28,160 | 4,056,715 | 27,350 | 3,954,935 |
| 4 | 173,372 | 33,091,924 | 75,907 | 14,130,092 | 25,172 | 3,585,173 | 24,474 | 3,495,562 |
| 5 | 962,139 | 181,517,936 | 272,430 | 51,472,828 | 163,724 | 20,975,496 | 159,188 | 20,404,260 |
| 6 | 193,464 | 36,498,873 | 61,315 | 11,507,854 | 34,060 | 4,829,387 | 33,194 | 4,717,419 |
| 7 | 138,979 | 25,873,364 | 43,694 | 8,112,493 | 23,660 | 3,338,544 | 23,060 | 3,263,342 |
| 8 | 108,513 | 20,432,086 | 35,802 | 6,732,367 | 21,300 | 3,028,530 | 20,700 | 2,955,317 |
| 9 | 140,580 | 27,337,739 | 64,072 | 12,282,317 | 36,750 | 5,267,569 | 36,270 | 5,210,008 |
| 10 | 175,930 | 32,252,210 | 69,015 | 12,447,271 | 28,538 | 3,964,211 | 27,658 | 3,861,508 |
| 11 | 124,820 | 24,432,874 | 46,345 | 8,596,991 | 24,546 | 3,539,762 | 24,066 | 3,479,666 |
| 12 | 231,318 | 43,723,390 | 64,935 | 12,174,183 | 34,522 | 4,960,985 | 33,570 | 4,841,727 |
| 13 | 107,228 | 21,526,500 | 46,919 | 8,829,809 | 21,680 | 3,122,091 | 21,388 | 3,086,266 |
| 14 | 135,280 | 26,684,971 | 35,029 | 6,542,377 | 22,754 | 3,296,072 | 21,978 | 3,204,833 |
| 15 | 94,516 | 18,126,677 | 33,586 | 6,279,564 | 15,530 | 2,252,453 | 15,250 | 2,217,910 |
| 16 | 122,185 | 22,942,577 | 45,657 | 8,632,510 | 32,050 | 4,664,134 | 31,542 | 4,600,827 |
| 17 | 145,514 | 28,282,667 | 87,456 | 16,515,574 | 32,102 | 4,511,126 | 31,840 | 4,480,662 |
| 18 | 133,743 | 25,276,157 | 47,740 | 8,905,724 | 27,878 | 4,062,575 | 27,480 | 4,014,468 |
| 19 | 70,119 | 13,820,613 | 39,991 | 7,633,120 | 20,470 | 2,945,069 | 20,312 | 2,926,451 |
| 20 | 129,271 | 23,979,356 | 35,450 | 6,685,403 | 24,624 | 3,555,910 | 23,868 | 3,463,030 |
| 21 | 155,516 | 29,524,756 | 37,875 | 7,229,423 | 27,064 | 3,900,506 | 26,108 | 3,785,052 |
| 22 | 130,856 | 25,123,162 | 82,757 | 15,758,581 | 27,072 | 3,868,145 | 26,804 | 3,833,995 |

Wherein “Read Number” indicates a number of sequences in the data set; and “Read Base” indicates a number of bases in the data set.

In S2, an immune group database, a genome database and a primer database are established.

Reproductive cell sequences from which recombination of TCRD in IMGT database originates is established as an immune database. There are two genes in a D gene of TCRD, namely TRDD1, TRDD2 and TRDD3, but a length of a TRDD gene is less than 20 bp, and a longest one is only 13 bp, so it is not used to construct the immune database. TRDV and TRDJ genes are used to construct the immune database, which are TRD-V and TRD-J, respectively.

The database is established using the GRCh38 version of the human genome, which is formatted by using the makeblastdb, and the formatted database is named GRCh38-db.

In S3, an amplification primer pair (upstream primer and downstream primer) is established into a database, which is formatted by using the makeblastdb, and the formatted database is named Primer-db.

The sequencing data is aligned to an established immune database. Alignment is identified. Description for identification and alignment criterion can be referred to S3. “Mapped” and “unmapped” items of “Assembled” and “Unassembled” sets are counted, see Table 3.

In S4, sequence de-redundancy processing and source identification are performed on the unmapped data set, so as to obtain a specific position of an identified sequence on the genome from which it originates. A method of de-redundancy is shown in S4.1, and a “clstr” data set after de-redundancy is counted, see Table 4. The results show that clustering effect of the “unmapped” data set of the “Assembled” set is obvious. With this analysis of 22 samples, at least 70% de-redundancy sequences can be achieved, and “clstr” of Top1 and Top5 contains a large proportion of sequences, which is a representative sample. Clustering effect of “unmapped” data set of the “Unassembled” set is obvious, and with this sample clustering, at least 65% de-redundancy sequences can be achieved, and “clstr” of Top1 and Top5 contains a small proportion of sequences, which is not a representative sample.

TABLE 4

| | Assembled Unmapped | | | | | Unassembled Unmapped | | | | |
|---|---|---|---|---|---|---|---|---|---|---|
| | Before | After | clstr | | | Before | After | clstr | | |
| | clstr | clstr | Ratio(%) | Top1 | Top 5 | clstr | clstr | Ratio(%) | Top1 | Top5 |
| 1 | 53926 | 11113 | 0.79 | 10723 | 23997 | 27746 | 5196 | 0.81 | 169 | 660 |
| 2 | 41424 | 10590 | 0.74 | 6873 | 16730 | 29422 | 5845 | 0.80 | 206 | 765 |
| 3 | 43196 | 10143 | 0.77 | 7136 | 15258 | 27350 | 6114 | 0.78 | 188 | 738 |
| 4 | 75907 | 11772 | 0.84 | 24803 | 49258 | 24474 | 5826 | 0.76 | 173 | 553 |
| 5 | 272430 | 130002 | 0.52 | 43053 | 85670 | 159188 | 60722 | 0.62 | 17989 | 34561 |
| 6 | 61315 | 16984 | 0.72 | 9010 | 20814 | 33194 | 9861 | 0.70 | 184 | 677 |
| 7 | 43694 | 11812 | 0.73 | 4892 | 14086 | 23060 | 5499 | 0.76 | 150 | 564 |
| 8 | 35802 | 8705 | 0.76 | 7130 | 16445 | 20700 | 6241 | 0.70 | 116 | 449 |
| 9 | 64072 | 14068 | 0.78 | 14581 | 30444 | 36270 | 8806 | 0.76 | 198 | 760 |
| 10 | 69015 | 16024 | 0.77 | 14488 | 28986 | 27658 | 7459 | 0.73 | 252 | 676 |
| 11 | 46345 | 8208 | 0.82 | 12949 | 27730 | 24066 | 7798 | 0.68 | 218 | 632 |
| 12 | 64935 | 11292 | 0.83 | 19022 | 37867 | 33570 | 7545 | 0.78 | 189 | 744 |
| 13 | 46919 | 6058 | 0.87 | 14789 | 31463 | 21388 | 6116 | 0.71 | 175 | 600 |
| 14 | 35029 | 5768 | 0.84 | 9094 | 23733 | 21978 | 5876 | 0.73 | 150 | 563 |
| 15 | 33586 | 5816 | 0.83 | 9514 | 19560 | 15250 | 3255 | 0.79 | 105 | 407 |
| 16 | 45657 | 9043 | 0.80 | 7444 | 22631 | 31542 | 5750 | 0.82 | 215 | 820 |
| 17 | 87456 | 15885 | 0.82 | 21270 | 48776 | 31840 | 9265 | 0.71 | 246 | 833 |
| 18 | 47740 | 5927 | 0.88 | 14041 | 31840 | 27480 | 7516 | 0.73 | 216 | 715 |
| 19 | 39991 | 9258 | 0.77 | 8213 | 17714 | 20312 | 7244 | 0.64 | 123 | 414 |
| 20 | 35450 | 7542 | 0.79 | 6461 | 17889 | 23868 | 4923 | 0.79 | 169 | 618 |
| 21 | 37875 | 9200 | 0.76 | 6821 | 14499 | 26108 | 6951 | 0.73 | 142 | 534 |
| 22 | 82757 | 14161 | 0.83 | 19288 | 47944 | 26804 | 8463 | 0.68 | 156 | 538 |

Wherein “Before clstr” indicates counted data volume before de-redundancy; “After clstr” indicates counted data volume after de-redundancy; “clstr Ratio(%)” indicates a percentage of de-redundancy data; “Top1” indicates a number of sequences in the largest clstr; Top5: a number of sequences in top 5 clstr.

By aligning the “clstr” data set to the GRCh38-db database, the sequence can be identified to be from a specific genome. A case where a sequence source position is identified is counted and analyzed, and what is counted includes a genome source with optimal alignment for respective sequences, a specific genome position where the sequence source is mainly concentrated. With a good alignment effect, an exact position on the genome can be determined. Location sites with most genomic loci in the Top5 of “clstr” data sets can be referred to Table 5, it is noted that the genomic loci mentioned in this table should be reduced in optimizing and designing of this set of multi-primers.

TABLE 5

| Chromosome | Start | End |
|---|---|---|
| 4 | 12651144 | 12650858 |
| 11 | 1386413 | 1386127 |
| 12 | 99429746 | 99430035 |
| 7 | 29099436 | 29099726 |
| 22 | 39573097 | 39572807 |
| 8 | 43150345 | 43150195 |
| 2 | 237917028 | 237916880 |
| 1 | 43010806 | 43010656 |
| 17 | 3930933 | 3931083 |
| 8 | 72229663 | 72229513 |
| 8 | 102779860 | 102780009 |
| 15 | 39677922 | 39677774 |
| 3 | 169809895 | 169810045 |
| 14 | 22163706 | 22163856 |
| 10 | 26882770 | 26882622 |
| 5 | 14395864 | 14396014 |
| 7 | 101715639 | 101715788 |
| 6 | 42788271 | 42788421 |
| 16 | 86748060 | 86748209 |
| 2 | 22681975 | 22681825 |

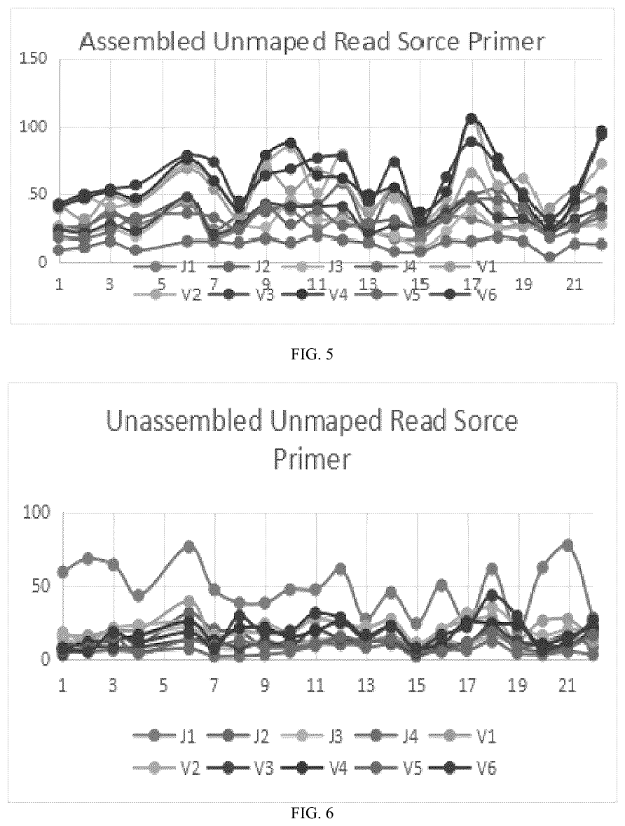

In S5, amplification primer sources of the top 5 of the clstr data set and the clstr data set are respectively identified. The “clstr” data set and the “clstr” data set are aligned to Primer-db, which was used to analyze the primer source of the data set (see FIG. 5, FIG. 6 and Table 6). The sequences in top5 of “clstr” data sets are aligned to the Primer-db, which was used to analyze the primer source of the data set. For the “clstr” data set of the “Assembled” data set, V4, V5 and V6 are the primer sequence sources. For the “clstr” data set of the “Ussembled”, V1 is a high-level primer sequence source. For top 5 data of the “clstr” data set, J1 and J3 are high-level primer sequence sources. FIG. 5 show a result of aligning the “clstr” data set in the “Assembled” data set to the Primer-db database, with an abscissa of the sample number and an ordinate of the number of the alignment. FIG. 6 show a result of aligning the “clstr” data set in the “Unassembled” to the Primer-db database, with an abscissa of the sample number and an ordinate of the number of the alignment.

TABLE 6

| | Assembled | | | | | | | | | | Unassembled Unmapped | | | | | | | | | |
|---|---|---|---|---|---|---|---|---|---|---|---|---|---|---|---|---|---|---|---|---|
| | J1 | J2 | J3 | J4 | V1 | V2 | V3 | V4 | V5 | V6 | J1 | J2 | J3 | J4 | V1 | V2 | V3 | V4 | V5 | V6 |
| 1 | 22 | 25 | 26 | 9 | 42 | 38 | 23 | 40 | 18 | 42 | 59 | 9 | 14 | 6 | 16 | 18 | 3 | 7 | 6 | 7 |
| 2 | 25 | 26 | 21 | 11 | 31 | 47 | 21 | 46 | 17 | 49 | 68 | 14 | 9 | 5 | 16 | 12 | 8 | 11 | 7 | 5 |
| 3 | 37 | 34 | 24 | 15 | 48 | 40 | 27 | 51 | 22 | 53 | 64 | 19 | 12 | 6 | 19 | 21 | 8 | 13 | 15 | 18 |
| 4 | 27 | 31 | 19 | 9 | 43 | 44 | 22 | 46 | 32 | 56 | 43 | 10 | 16 | 5 | 20 | 23 | 8 | 16 | 4 | 12 |
| 5 | 1093 | 1028 | 1223 | 467 | 1701 | 1337 | 1382 | 2772 | 976 | 966 | 561 | 242 | 306 | 108 | 410 | 311 | 296 | 548 | 254 | 241 |
| 6 | 42 | 45 | 45 | 15 | 68 | 71 | 47 | 75 | 35 | 78 | 76 | 31 | 21 | 7 | 39 | 25 | 13 | 25 | 13 | 18 |
| 7 | 17 | 23 | 21 | 15 | 53 | 59 | 20 | 59 | 32 | 73 | 47 | 20 | 8 | 2 | 14 | 16 | 12 | 12 | 9 | 7 |
| 8 | 24 | 33 | 26 | 14 | 32 | 40 | 26 | 39 | 23 | 44 | 38 | 18 | 7 | 2 | 18 | 18 | 8 | 21 | 12 | 29 |
| 9 | 39 | 36 | 25 | 17 | 69 | 71 | 42 | 78 | 40 | 63 | 38 | 9 | 13 | 3 | 18 | 24 | 11 | 21 | 8 | 18 |
| 10 | 37 | 41 | 45 | 14 | 52 | 84 | 40 | 87 | 27 | 68 | 47 | 12 | 8 | 5 | 18 | 18 | 9 | 15 | 8 | 19 |
| 11 | 24 | 42 | 31 | 19 | 66 | 50 | 40 | 63 | 37 | 76 | 47 | 11 | 13 | 9 | 27 | 28 | 20 | 19 | 10 | 31 |
| 12 | 33 | 57 | 30 | 16 | 59 | 79 | 40 | 61 | 26 | 77 | 61 | 10 | 14 | 13 | 24 | 24 | 14 | 25 | 15 | 28 |
| 13 | 23 | 25 | 21 | 14 | 36 | 40 | 21 | 49 | 28 | 44 | 27 | 11 | 13 | 8 | 21 | 22 | 12 | 14 | 12 | 16 |
| 14 | 17 | 53 | 19 | 8 | 54 | 46 | 26 | 54 | 30 | 73 | 45 | 15 | 14 | 10 | 27 | 26 | 12 | 22 | 13 | 22 |
| 15 | 17 | 23 | 11 | 8 | 29 | 29 | 25 | 36 | 23 | 29 | 24 | 8 | 9 | 2 | 7 | 11 | 4 | 7 | 6 | 7 |
| 16 | 32 | 38 | 22 | 15 | 35 | 46 | 35 | 51 | 31 | 62 | 50 | 10 | 13 | 7 | 20 | 20 | 7 | 10 | 5 | 16 |
| 17 | 31 | 48 | 37 | 15 | 65 | 105 | 46 | 105 | 45 | 88 | 24 | 10 | 10 | 6 | 26 | 31 | 11 | 25 | 11 | 22 |
| 18 | 25 | 53 | 24 | 18 | 39 | 56 | 32 | 76 | 45 | 70 | 61 | 24 | 15 | 12 | 28 | 35 | 21 | 24 | 18 | 43 |
| 19 | 27 | 31 | 25 | 15 | 26 | 61 | 31 | 46 | 39 | 50 | 19 | 8 | 11 | 4 | 13 | 23 | 12 | 23 | 8 | 29 |
| 20 | 30 | 20 | 24 | 4 | 39 | 32 | 22 | 24 | 18 | 31 | 62 | 11 | 12 | 3 | 26 | 16 | 9 | 10 | 5 | 6 |
| 21 | 26 | 40 | 25 | 13 | 52 | 47 | 31 | 45 | 24 | 51 | 77 | 16 | 13 | 5 | 27 | 20 | 9 | 15 | 8 | 12 |
| 22 | 38 | 51 | 27 | 13 | 46 | 72 | 39 | 96 | 33 | 93 | 28 | 8 | 16 | 3 | 12 | 19 | 20 | 22 | 16 | 26 |

Of course, the above is only schematic description, and specific data can be set according to actual needs, which is not limited herein.

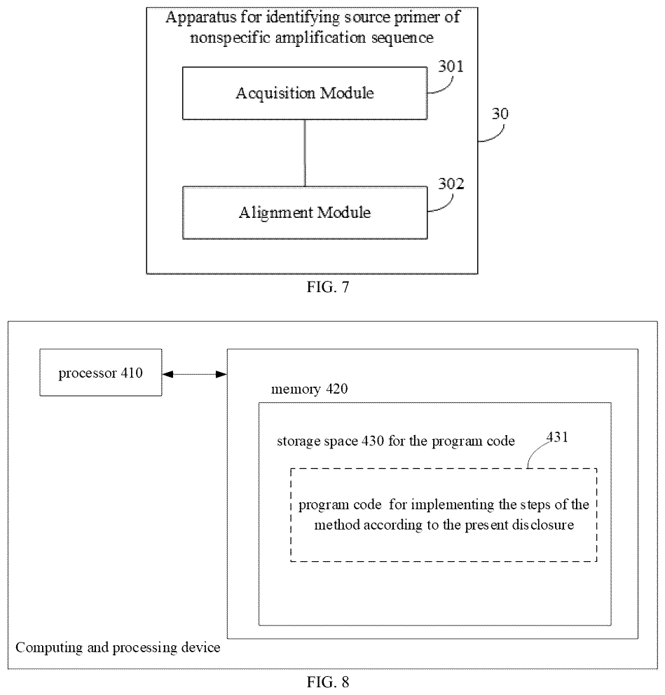

FIG. 7 schematically shows a structural diagram of an apparatus 30 for identifying a source primer of a nonspecific amplification sequence according to the present disclosure. The apparatus includes an acquisition module 301 and an alignment module 302.

The acquisition module 301 is configured to acquire amplification sequence data of an amplified gene obtained by primer amplification of a target gene fragment, source gene sequence data of a source gene to which the target gene fragment belongs, and primer sequence data used in the primer amplification.

The alignment module 302 is configured to align the amplification sequence data to the source gene sequence data, and to take the amplification sequence data that does not match the source gene sequence data as nonspecific amplification sequence data; and
align the nonspecific amplification sequence data to the primer sequence data, and to take a primer with the primer sequence data being matched with the nonspecific amplification sequence data as an amplification source primer of the nonspecific amplification sequence.

Optionally, the alignment module 302 is further configured for:
aligning the nonspecific amplification sequence data to reference genome sequence data to obtain an alignment result; and
determining position information of source gene of the nonspecific amplification sequence on the genome according to the alignment result.

Optionally, the alignment module 302 is further configured for:
counting at least one of a genome source, a sequence position and sequence features of the nonspecific amplification sequence on the reference genome according to distribution and a position of the alignment result on the reference genome sequence data.

Optionally, when the target gene fragment is an immune gene fragment, the gene sequence data includes sequence data with overlapping double-ended sequencing and sequence data with non-overlapping double-ended sequencing;

Optionally, the acquisition module 301 is further configured for:
acquiring raw data obtained by primer amplification of the gene fragment;
performing an overlapping operation on a first gene fragment and a second gene fragment in the raw data with an overlapping sequence length being greater than or equal to a first sequence length threshold and with a sequence length after overlapping being greater than or equal to a second sequence length threshold, so as to obtain the sequence data with overlapping double-ended sequencing; and
taking amplification sequence data in the raw data with an overlapping sequence length being less than the first sequence length threshold or with a sequence length after overlapping being less than the second sequence length threshold as the sequence data with non-overlapping double-ended sequencing.

Optionally, the source gene sequence data includes sequence data in a V gene family, sequence data in a D gene family and sequence data in a J gene family;

Optionally, the alignment module 302 is further configured for:
aligning the sequence data with overlapping double-ended sequencing and the sequence data with non-overlapping double-ended sequencing to the sequence data in the V gene family, the sequence data in the D gene family and the sequence data in the J gene family, respectively, so as to obtain a consistency alignment value of the sequence data with overlapping double-ended sequencing and the sequence data with non-overlapping double-ended sequencing;
taking a sum of lengths of sequence data in the sequence data with overlapping double-ended sequencing with consistency alignment values to the sequence data in the V gene family, the sequence data in the D gene family and the sequence data in the J gene family being greater than or equal to a consistency alignment value threshold as a comparable length of the sequence data with overlapping double-ended sequencing;
taking the sequence data with overlapping double-ended sequencing with the comparable length being less than a comparable length threshold as the nonspecific amplification sequence data; and
taking sequence data in the sequence data with non-overlapping double-ended sequencing with consistency alignment values to the sequence data in the V gene family, the sequence data in the D gene family and the sequence data in the J gene family being smaller than the consistency alignment value threshold as the nonspecific amplification sequence data.

Optionally, the alignment module 302 is further configured for:
performing de-redundancy processing on the nonspecific amplification sequence data, so as to remove redundant sequence data of the nonspecific amplification sequence data, the redundant sequence data being sequence data with a proportion of repeated bases in the sequence being greater than or equal to a proportion threshold.

Optionally, the acquisition module 301 is further configured for:
removing low-quality sequence data in the amplification sequence data.

Optionally, the acquisition module 301 is further configured for:
removing adapter sequence data with a sequence end length being greater than or equal to an end length threshold in the amplification sequence data after being spliced, and removing sequence data with a sequence average quality value being less than a quality value threshold in the amplification sequence data.

Optionally, the acquisition module 301 is further configured for:
removing a low-quality section with the quality value being less than the quality value threshold in the amplification sequence data, and removing the low-quality sequence data with a sequence length being less than a third sequence length threshold in the amplification sequence data after the low-quality section being removed.

In the embodiment of the present disclosure, the amplification sequence data of the amplified gene is aligned to the source gene sequence data from which the primer amplification target gene fragment originates to screen out the nonspecific amplification sequence data in the amplification sequence data, and then the nonspecific amplification sequence data is aligned to the primer sequence data of the primer used in amplification to screen out the primer sequence data matched with the nonspecific amplification sequence data, so that the amplification source primer of the nonspecific amplification sequence in the amplification sequence can be accurately identified.

The embodiments of each component in the present disclosure can be implemented by hardware, or by software modules running on one or more processors, or by their combination. A person skilled in the art should understand that the microprocessor or digital signal processor (DSP) can be used in practice to realize some or all functions of some or all components in the calculation and processing equipment according to the embodiments of the present disclosure the present disclosure. The present disclosure can also be implemented as the equipment or device programs (for example, computer programs and computer program products) used to execute part or all of the methods described here. The programs of implementing the present disclosure may be stored in a computer-readable medium, or can have the form of one or more signals. Such signals can be downloaded from the Internet site, or provided on the carrier signal, or provided in any other form.

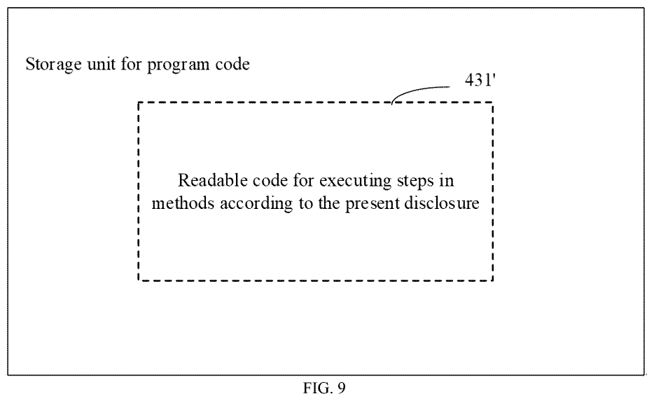

For example, FIG. 8 shows a calculating and processing device that can implement the method according to the present disclosure. The calculating and processing device traditionally includes a processor 410 and a computer program product or computer-readable medium in the form of a memory 420. The memory 420 may be electronic memories such as flash memory, EEPROM (Electrically Erasable Programmable Read Only Memory), EPROM, hard disk or ROM. The memory 420 has the storage space 430 of the program code 431 for implementing any steps of the above method. For example, the storage space 430 for program code may contain program codes 431 for individually implementing each of the steps of the above method. Those program codes may be read from one or more computer program products or be written into the one or more computer program products. Those computer program products include program code carriers such as a hard disk, a compact disk (CD), a memory card or a floppy disk. Such computer program products are usually portable or fixed storage units as shown in FIG. 9. The storage unit may have storage segments or storage spaces with similar arrangement to the memory 420 of the calculating and processing device in FIG. 8. The program codes may, for example, be compressed in a suitable form. Generally, the storage unit contains a computer-readable code 431′, which can be read by a processor like 410. When those codes are executed by the calculating and processing device, the codes cause the calculating and processing device to implement each of the steps of the method described above.

It should be understood that although the steps in the flow chart of the figures are displayed in turn according to the instructions of the arrows, these steps are not necessarily performed in the order indicated by the arrows. Unless this article makes it clear that there are no strict order restrictions on the execution of these steps, they can be executed in other order. Moreover, at least part of the steps in the flow chart can include multiple sub-steps or multiple stages. These sub-steps or stages are not necessarily completed at the same time, but can be executed at different times. The order of execution is not necessarily sequential, but can be performed by taking turns or alternately with at least part of sub-steps of other steps or stages of other steps.

The term “one embodiment”, “an embodiment”, or “one or more embodiments” herein means that the particular features, structures, or features described in combination with embodiments are included in at least one embodiment disclosed herein. Also, note that the examples of words “in an embodiment” here do not necessarily all refer to the same embodiment.

A great deal of detail is provided in the manual provided here. However, it is understood that this disclosed embodiment can be practiced without such specific details. In some instances, known methods, structures and techniques are not detailed so as not to obscure the understanding of this specification.

In a claim, no reference symbol between parentheses shall be constructed to restrict the claim. The word “include” does not exclude the existence of elements or steps not listed in the claim. The word “one” or “one” before a component does not preclude the existence of more than one such component. This exposure can be implemented with the help of hardware including several different components and with the help of properly programmed computers. In listing the unit claims of several devices, several of these devices can be embodied by the same hardware item. The use of the words first, second, and third does not indicate any order. These words can be interpreted as names.

Finally, it should be noted that the above embodiments are only used to illustrate, and not to limit, the disclosed technical solution; notwithstanding the detailed description of the present disclosure with reference to the foregoing embodiments, ordinary technical personnel in the field should understand that they can still modify the technical solutions recorded in the foregoing embodiments, or make equivalent substitutions to some of the technical features thereof; such modifications or substitutions shall not separate the essence of the corresponding technical solutions from the spirit and scope of the technical solutions of the disclosed embodiments.

1. A method for identifying a source primer of a nonspecific amplification sequence, comprising:
acquiring amplification sequence data of an amplified gene obtained by primer amplification of a target gene fragment, source gene sequence data of a source gene to which the target gene fragment belongs, and primer sequence data used in the primer amplification;
aligning the amplification sequence data to the source gene sequence data, and taking the amplification sequence data that does not match the source gene sequence data as nonspecific amplification sequence data; and
aligning the nonspecific amplification sequence data to the primer sequence data, and taking a primer with the primer sequence data being matched with the nonspecific amplification sequence data as an amplification source primer of the nonspecific amplification sequence.

2. The method according to claim 1, wherein when the target gene fragment is an immune gene fragment, the gene sequence data comprises sequence data with overlapping double-ended sequencing and sequence data with non-overlapping double-ended sequencing;
acquiring the amplification sequence data of the amplified gene obtained by primer amplification of the target gene fragment comprises:
acquiring raw data obtained by primer amplification of the gene fragment;
performing an overlapping operation on a first gene fragment and a second gene fragment in the raw data with an overlapping sequence length being greater than or equal to a first sequence length threshold and with a sequence length after overlapping being greater than or equal to a second sequence length threshold, so as to obtain the sequence data with overlapping double-ended sequencing; and
taking amplification sequence data in the raw data with an overlapping sequence length being less than the first sequence length threshold or with a sequence length after overlapping being less than the second sequence length threshold as the sequence data with non-overlapping double-ended sequencing.

3. The method according to claim 2, wherein the source gene sequence data comprises sequence data in a V gene family, sequence data in a D gene family and sequence data in a J gene family;
aligning the amplification sequence data to the source gene sequence data, and taking the amplification sequence data that does not match the source gene sequence data as the nonspecific amplification sequence data comprises:
aligning the sequence data with overlapping double-ended sequencing and the sequence data with non-overlapping double-ended sequencing to the sequence data in the V gene family, the sequence data in the D gene family and the sequence data in the J gene family, respectively, so as to obtain a consistency alignment value of the sequence data with overlapping double-ended sequencing and the sequence data with non-overlapping double-ended sequencing;
taking a sum of lengths of sequence data in the sequence data with overlapping double-ended sequencing with consistency alignment values to the sequence data in the V gene family, the sequence data in the D gene family and the sequence data in the J gene family being greater than or equal to a consistency alignment value threshold as a comparable length of the sequence data with overlapping double-ended sequencing;
taking the sequence data with overlapping double-ended sequencing with the comparable length being less than a comparable length threshold as the nonspecific amplification sequence data; and
taking sequence data in the sequence data with non-overlapping double-ended sequencing with consistency alignment values to the sequence data in the V gene family, the sequence data in the D gene family and the sequence data in the J gene family being smaller than the consistency alignment value threshold as the nonspecific amplification sequence data.

4. The method according to claim 1, wherein after aligning the amplification sequence data to the source gene sequence data, and taking the amplification sequence data that does not match the source gene sequence data as the nonspecific amplification sequence data, the method further comprises:
aligning the nonspecific amplification sequence data to reference genome sequence data to obtain an alignment result; and
determining position information of source gene of the nonspecific amplification sequence on the genome according to the alignment result.

5. The method according to claim 4, wherein determining the position information of the source gene of the nonspecific amplification sequence on the genome according to the alignment result comprises:
counting at least one of a genome source, a sequence position and sequence features of the nonspecific amplification sequence on the reference genome according to distribution and a position of the alignment result on the reference genome sequence data.

6. The method according to claim 1, wherein after aligning the amplification sequence data to the source gene sequence data, and taking the amplification sequence data that does not match the source gene sequence data as the nonspecific amplification sequence data, the method further comprises:
performing de-redundancy processing on the nonspecific amplification sequence data, so as to remove redundant sequence data of the nonspecific amplification sequence data, the redundant sequence data being sequence data with a proportion of repeated bases in the sequence being greater than or equal to a proportion threshold.

7. The method according to claim 1, wherein before aligning the amplification sequence data to the source gene sequence data, and taking the amplification sequence data that does not match the source gene sequence data as the nonspecific amplification sequence data, the method further comprises:
removing low-quality sequence data in the amplification sequence data.

8. The method according to claim 7, wherein removing the low-quality sequence data in the amplification sequence data comprises:
removing adapter sequence data with a sequence end length being greater than or equal to an end length threshold in the amplification sequence data after being spliced, and removing sequence data with a sequence average quality value being less than a quality value threshold in the amplification sequence data.

9. The method according to claim 7, wherein removing the low-quality sequence data in the amplification sequence data comprises:
removing a low-quality section with the quality value being less than the quality value threshold in the amplification sequence data, and removing the low-quality sequence data with a sequence length being less than a third sequence length threshold in the amplification sequence data after the low-quality section being removed.

10. (canceled)

11. A computing processing device, comprising:
a memory with computer-readable code stored therein;
one or more processors, the computing processing device executing the method for identifying the source primer of the nonspecific amplification sequence according to claim 1 when the computer-readable code is executed by the one or more processors.

12. (canceled)

13. A non-transient computer-readable medium with a computer program of the method for identifying the source primer of the nonspecific amplification sequence according to claim 1 stored therein.

14. The computing processing device according to claim 11, wherein when the target gene fragment is an immune gene fragment, the gene sequence data comprises sequence data with overlapping double-ended sequencing and sequence data with non-overlapping double-ended sequencing;
acquiring the amplification sequence data of the amplified gene obtained by primer amplification of the target gene fragment comprises:
acquiring raw data obtained by primer amplification of the gene fragment;
performing an overlapping operation on a first gene fragment and a second gene fragment in the raw data with an overlapping sequence length being greater than or equal to a first sequence length threshold and with a sequence length after overlapping being greater than or equal to a second sequence length threshold, so as to obtain the sequence data with overlapping double-ended sequencing; and
taking amplification sequence data in the raw data with an overlapping sequence length being less than the first sequence length threshold or with a sequence length after overlapping being less than the second sequence length threshold as the sequence data with non-overlapping double-ended sequencing.

15. The computing processing device according to claim 14, wherein the source gene sequence data comprises sequence data in a V gene family, sequence data in a D gene family and sequence data in a J gene family;
aligning the amplification sequence data to the source gene sequence data, and taking the amplification sequence data that does not match the source gene sequence data as the nonspecific amplification sequence data comprises:
aligning the sequence data with overlapping double-ended sequencing and the sequence data with non-overlapping double-ended sequencing to the sequence data in the V gene family, the sequence data in the D gene family and the sequence data in the J gene family, respectively, so as to obtain a consistency alignment value of the sequence data with overlapping double-ended sequencing and the sequence data with non-overlapping double-ended sequencing;
taking a sum of lengths of sequence data in the sequence data with overlapping double-ended sequencing with consistency alignment values to the sequence data in the V gene family, the sequence data in the D gene family and the sequence data in the J gene family being greater than or equal to a consistency alignment value threshold as a comparable length of the sequence data with overlapping double-ended sequencing;
taking the sequence data with overlapping double-ended sequencing with the comparable length being less than a comparable length threshold as the nonspecific amplification sequence data; and
taking sequence data in the sequence data with non-overlapping double-ended sequencing with consistency alignment values to the sequence data in the V gene family, the sequence data in the D gene family and the sequence data in the J gene family being smaller than the consistency alignment value threshold as the nonspecific amplification sequence data.

16. The computing processing device according to claim 11, wherein after aligning the amplification sequence data to the source gene sequence data, and taking the amplification sequence data that does not match the source gene sequence data as the nonspecific amplification sequence data, the method further comprises:
aligning the nonspecific amplification sequence data to reference genome sequence data to obtain an alignment result; and
determining position information of source gene of the nonspecific amplification sequence on the genome according to the alignment result.

17. The computing processing device according to claim 16, wherein determining the position information of the source gene of the nonspecific amplification sequence on the genome according to the alignment result comprises:
counting at least one of a genome source, a sequence position and sequence features of the nonspecific amplification sequence on the reference genome according to distribution and a position of the alignment result on the reference genome sequence data.

18. The computing processing device according to claim 11, wherein after aligning the amplification sequence data to the source gene sequence data, and taking the amplification sequence data that does not match the source gene sequence data as the nonspecific amplification sequence data, the method further comprises:
performing de-redundancy processing on the nonspecific amplification sequence data, so as to remove redundant sequence data of the nonspecific amplification sequence data, the redundant sequence data being sequence data with a proportion of repeated bases in the sequence being greater than or equal to a proportion threshold.

19. The computing processing device according to claim 11, wherein before aligning the amplification sequence data to the source gene sequence data, and taking the amplification sequence data that does not match the source gene sequence data as the nonspecific amplification sequence data, the method further comprises:
removing low-quality sequence data in the amplification sequence data.

20. The computing processing device according to claim 19, wherein removing the low-quality sequence data in the amplification sequence data comprises:
removing adapter sequence data with a sequence end length being greater than or equal to an end length threshold in the amplification sequence data after being spliced, and removing sequence data with a sequence average quality value being less than a quality value threshold in the amplification sequence data.

21. The computing processing device according to claim 19, wherein removing the low-quality sequence data in the amplification sequence data comprises:
removing a low-quality section with the quality value being less than the quality value threshold in the amplification sequence data, and removing the low-quality sequence data with a sequence length being less than a third sequence length threshold in the amplification sequence data after the low-quality section being removed.

22. The non-transient computer-readable medium according to claim 13, wherein when the target gene fragment is an immune gene fragment, the gene sequence data comprises sequence data with overlapping double-ended sequencing and sequence data with non-overlapping double-ended sequencing;
acquiring the amplification sequence data of the amplified gene obtained by primer amplification of the target gene fragment comprises:
acquiring raw data obtained by primer amplification of the gene fragment;
performing an overlapping operation on a first gene fragment and a second gene fragment in the raw data with an overlapping sequence length being greater than or equal to a first sequence length threshold and with a sequence length after overlapping being greater than or equal to a second sequence length threshold, so as to obtain the sequence data with overlapping double-ended sequencing; and
taking amplification sequence data in the raw data with an overlapping sequence length being less than the first sequence length threshold or with a sequence length after overlapping being less than the second sequence length threshold as the sequence data with non-overlapping double-ended sequencing.
